(19) United States
(12) Patent Application Publication
(10) Pub. No.: US 2014/0336706 A1
(43) Pub. Date: Nov. 13, 2014

(54) TRANVERSE VERTEBRAL CONNECTOR

(71) Applicant: Laszlo GARAMSZEGI, Mission Viejo, CA (US)

(72) Inventor: Laszlo GARAMSZEGI, Mission Viejo, CA (US)

(21) Appl. No.: 13/890,136

(22) Filed: May 8, 2013

(51) Int. Cl. A61B 17/70

(52) U.S. Cl. CPC A61B 17/7052
USPC 606/252

(57) ABSTRACT

A transconnector is adapted to connect first and second spinal rods that are positioned longitudinally along a spine. The transconnector includes a first member and a second member that clamp onto first and second spinal rods.

### REFERENCE TO PRIORITY DOCUMENT

This application claims priority of co-pending U.S. Provisional Patent Application Ser. No. 60/898,010 filed Mar. 9, 2007. Priority of the aforementioned filing date is hereby claimed and the disclosures of the Provisional patent application is hereby incorporated by reference in its entirety.

### BACKGROUND

The use of spinal rods is conventional for correction of spinal trauma or conditions, such as curvature of the spine. Generally, an orthopedic stabilization system may include a pair of elongate members, such as spinal rods or plates, that are coupled to a bone or bones. For the sake of simplicity, the term “rod” is used throughout to refer to any elongate member. The rods are generally contoured and longitudinally disposed adjacent to vertebral bodies of a spine.

The strength and stability of the rod assembly can be increased by coupling the two rods with a cross-connector that extends substantially horizontal to the longitudinal axes of the rods across the spine. In some situations, the two rods are geometrically aligned such that the two rods are parallel to each other. However, the two rods are often not three dimensionally geometrically aligned in actual situations. There are several ways to address the variations of geometrical alignment. First, one or both of the rods can be bent to accommodate the transconnector. However, any bending in either of the rods can adversely affect the fixation to the spine and comprise clinical outcome. Furthermore, the bending can also adversely affect the mechanical properties of the rods. The transconnector can also be bent so that the disturbance to the rod positioning is minimized. As is the case with bending of the rods, the mechanical properties of the transconnector could be compromised.

Because of the forces acting along the transverse connector and the movement of the spinal rods, the connection between the transverse connector and the rod must be secure to avoid movement of the transverse connector along the spinal rod. Some rod fastening systems of transverse connectors use threaded fasteners to attach the transverse connector to adjacent rods. The threaded fastener can be a set screw or a nut. Not tightening a threaded fastener enough may allow movement of the transverse connector. Overtightening a threaded fastener could result in damage to the system and failure of the transverse connector.

### SUMMARY

In view of the foregoing, there exists a need for an improved transconnector for coupling spinal rods. Disclosed is a transconnector for connecting first and second spinal rods that are positioned longitudinally along a spine. The transconnector comprises a first member having a first clamp member adapted to clamp onto a first spinal rod, the first member having a first connecting region extending away from the clamp member; a second member having a second clamp member adapted to clamp onto a second spinal rod, the second member having a second connecting region extending away from the second clamp member toward the first member, wherein the second member and first member are slidably interconnected along the first and second connecting regions; a first interference pin coupled to the first clamp member and adapted to provide an interfering engagement with the first spinal rod to lock the first spinal rod in the first clamp member; a second interference pin coupled to the second clamp member and adapted to provide an interfering engagement with the second spinal rod to lock the second spinal rod in the second clamp member; and a third interference pin coupled to the first and second connecting regions and adapted to provide an interfering engagement between the first and second connecting regions to lock the first and second members in a fixed position relative to one another.

Other features and advantages will be apparent from the following description of various embodiments, which illustrate, by way of example, the principles of the disclosed devices and methods.

### BRIEF DESCRIPTION OF THE DRAWINGS

FIG. 1 shows a schematic of three vertebrae connected by longitudinal members and two transverse connectors.

FIG. 2 shows a perspective view of an embodiment of a transverse connector and two longitudinal members.

FIG. 3 shows an exploded view of the transverse connector of FIG. 2.

FIG. 4 shows a cross-sectional view of the transverse connector of FIG. 2.

FIG. 5 shows a top plan view of the transverse connector of FIG. 2.

FIG. 6 shows a perspective view of another embodiment of a transverse connector and two longitudinal members.

FIG. 7 shows an exploded view of the transverse connector of FIG. 6.

FIG. 8 shows a cross-sectional view of the transverse connector of FIG. 6.

FIG. 9 shows a top plan view of the transverse connector of FIG. 6.

FIG. 10 shows a perspective view of another embodiment of a transverse connector and two longitudinal members.

FIG. 11 shows an exploded view of the transverse connector of FIG. 10.

FIG. 12 shows a cross-sectional view of the transverse connector of FIG. 10.

FIG. 13 shows a top plan view of the transverse connector of FIG. 10.

FIG. 14 shows a perspective view of another embodiment of a transverse connector and two longitudinal members.

FIG. 15 shows an exploded view of the transverse connector of FIG. 14.

FIG. 16 shows a cross-sectional view of the transverse connector of FIG. 14.

FIG. 17 shows a top plan view of the transverse connector of FIG. 14.

FIG. 18 shows a perspective view of another embodiment of a transverse connector.

FIG. 19 shows an exploded view of the transverse connector of FIG. 18.

FIG. 20 shows a cross-sectional view of the transverse connector of FIG. 18.

### DETAILED DESCRIPTION

Disclosed herein are methods and devices for interconnecting first and second longitudinal members extending along a spinal column of a patient.

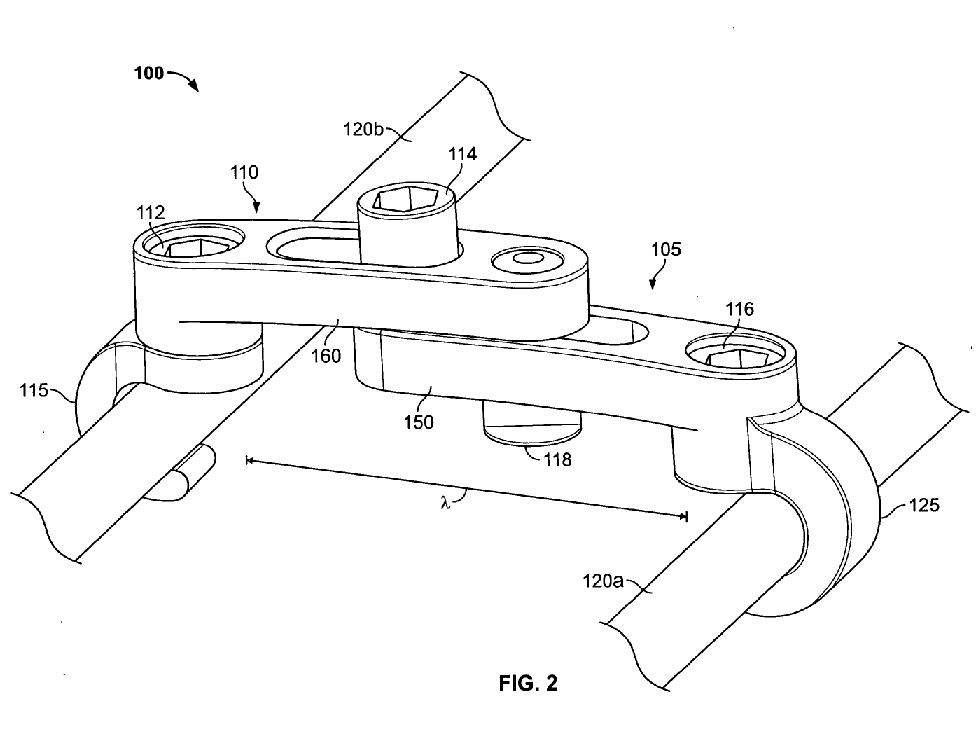

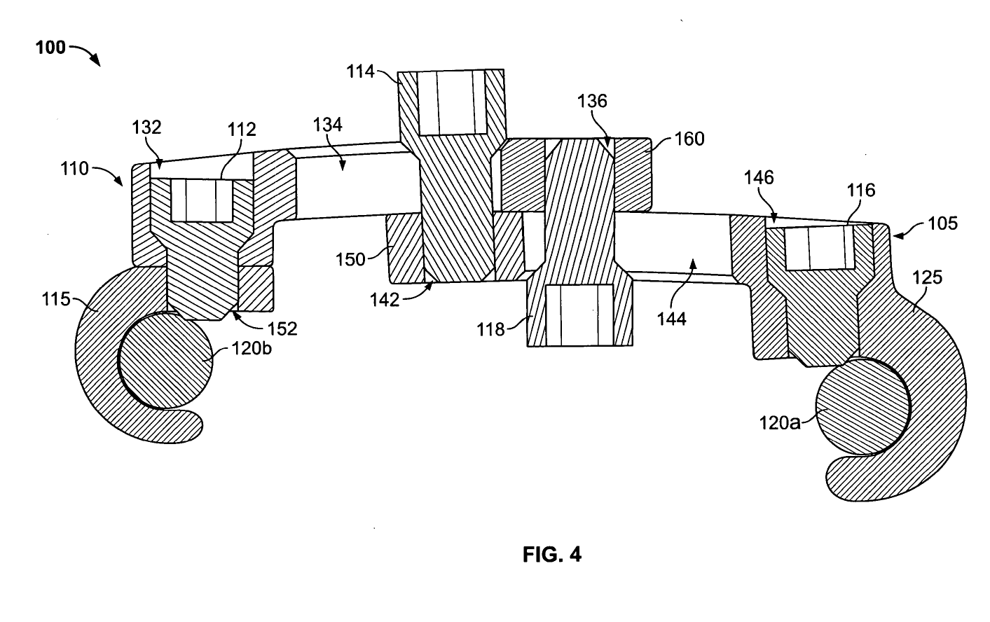

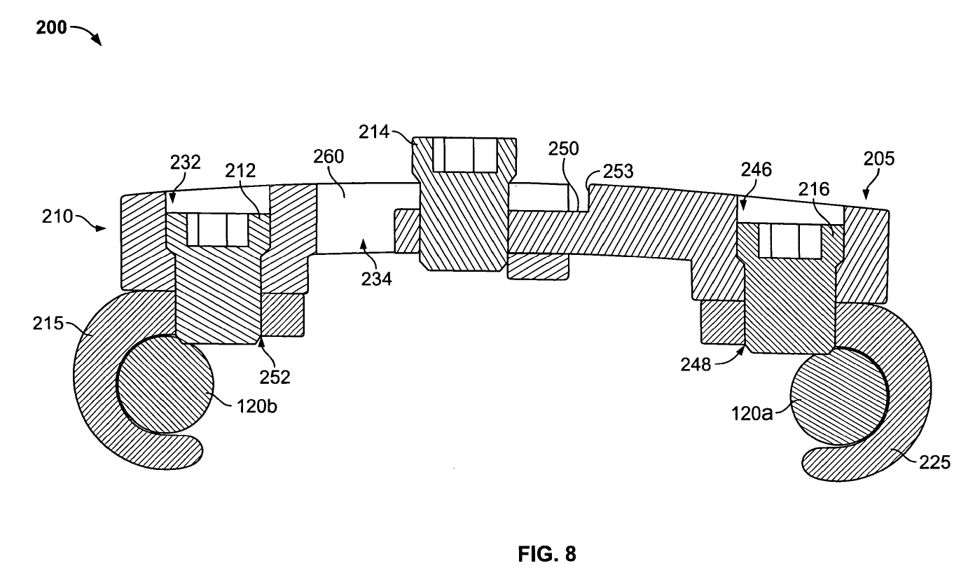

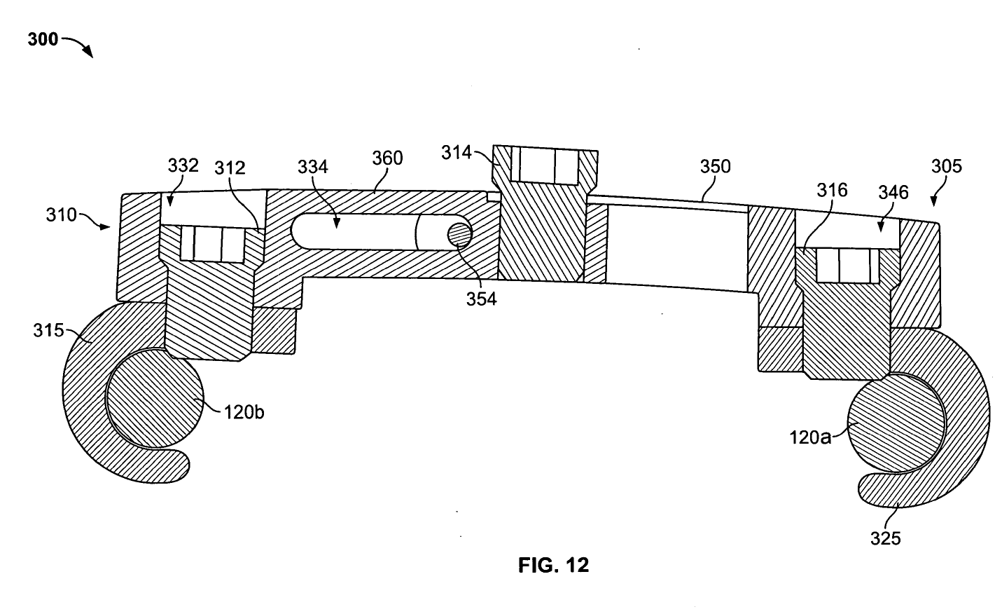

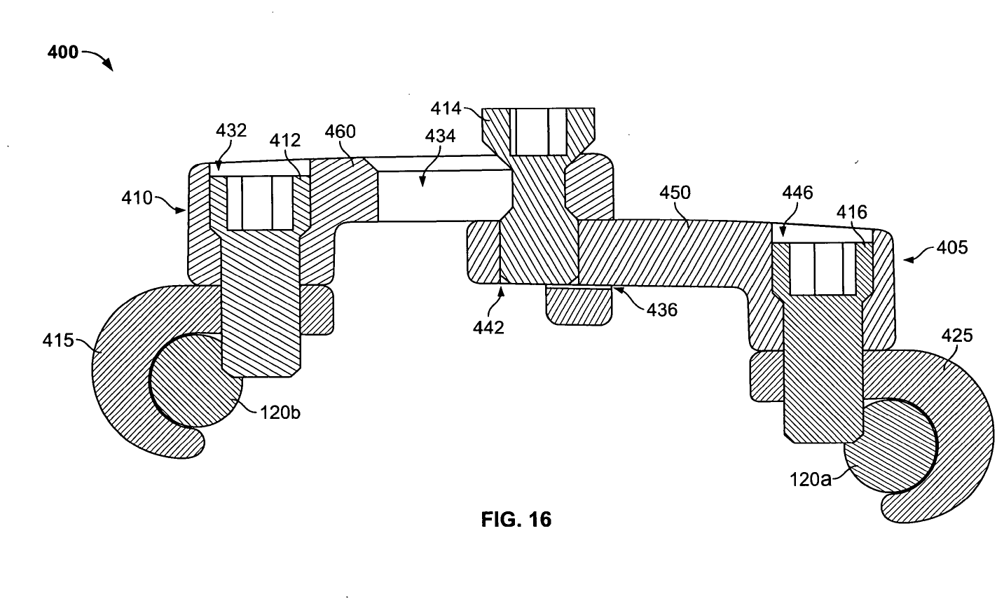

FIGS. 2-17 illustrate various embodiments of exemplary transverse connectors 100, 200, 300 and 400, and in each of the illustrated embodiments the transverse connector 100, 200, 300, 400 generally includes a first connecting member 105, 205, 305, 405 and a second connecting member 110, 210, 310, 410. Each pair of connecting member 105, 205, 305, 405, 110, 210, 310, 410 connect to each other transversely (i.e., across the midline of the vertebral column) in a variety of configurations. Each pair of connecting members 105, 205, 305, 405, 110, 210, 310, 410 are fixed to each other by a variety mechanisms generally involving a set screw 114, 118, 214, 314, 414. Further, each embodiment of the transverse connector 100, 200, 300 and 400 includes a clamping mechanism that is adapted to selectively lock a longitudinal member extending along a spinal column of a patient, such as a spinal fixation element, and in particular a spinal fixation rod. The fixation of the connecting member pairs and the clamping mechanisms allow for transverse as well as rotational adjustability of the transverse connectors 100, 200, 300 and 400.

A person skilled in the art will appreciate that while each transverse connector 100, 200, 300, 400 is described herein as being adapted to engage a spinal fixation element, and in particular a spinal fixation rod, that a transverse connector disclosed herein can be configured to engage a variety of spinal fixation devices, such as anchors, cables, fixation plates, etc. Moreover, the transverse connectors can include only one connector member for engaging a spinal fixation device, and the opposed terminal end of the transverse connectors can be adapted for other uses. For example, the opposed terminal end of the transverse connectors can be configured to be fixedly attached to a vertebra. The transverse connectors disclosed herein can also include any combination of features described and/or illustrated herein, and the transverse connectors are not limited to the illustrated embodiments.

As indicated above, the transverse connector in certain exemplary embodiments includes a first connecting member and a second connecting member that extend toward each other between each longitudinal member or spinal rod. The first and second connecting members can connect by a variety of mechanisms or configurations. The first and second connecting members can be generally elongate and positioned a distance apart from one another and adjusted transversely. The first and second connecting members can also be rotationally adjustable to allow the connecting members to be positioned as desired. The transverse and rotational adjustability of the transverse connectors allows them to mate to parallel, non-parallel, diverging, and converging spinal rods that are implanted within a patient's spinal system.

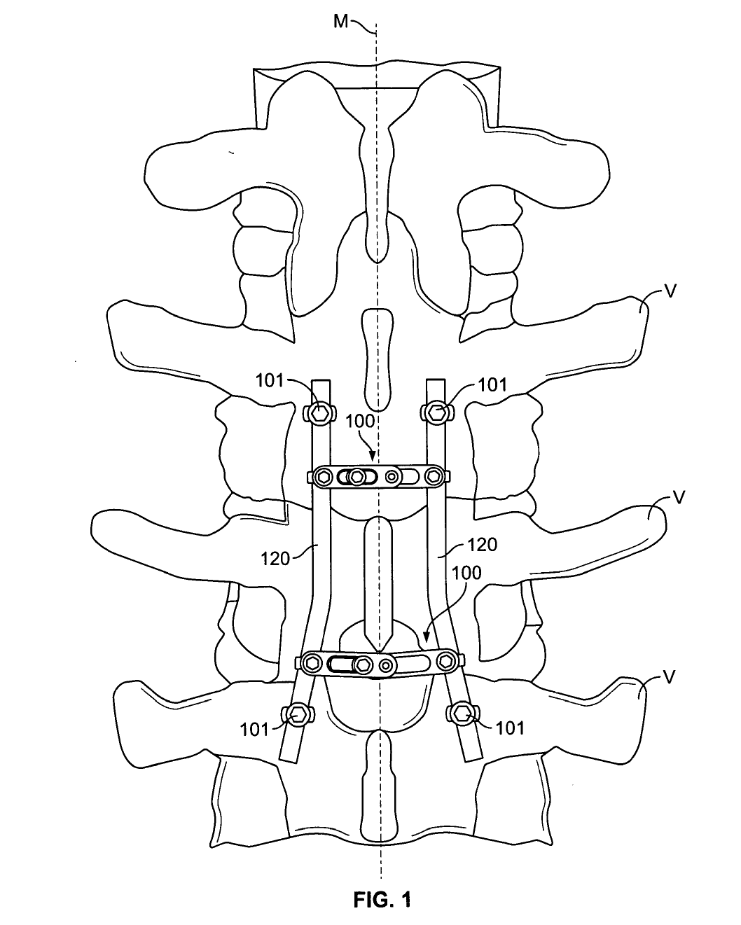

FIG. 1 shows an exemplary transverse connector 100 for interconnecting a pair of longitudinal members or rods 120 connected to vertebrae V of a spinal column. Although the transverse connector 100 is shown interconnecting rods 120, it is contemplated that the transverse connector 100 may interconnect any suitable longitudinal member, such as plates or rods of other shapes, such as hexagonal rods. The rods 120 can be located anywhere along the spinal column. The location of the rods illustrated in FIG. 1 is for example purposes.

Each of the rods 120 (FIG. 1) is elongate and has a sufficient length to span at least two vertebrae V. A plurality of connectors 101 connect the rods 120 with the vertebrae V. The connectors 101 can be of any known or desired configuration. In an embodiment, the connectors 101 are pedicle screw assemblies that include a receiver member that removably mates with a screw member in a poly-axial or mono-axial configuration.

At least one transverse connector 100 (FIG. 1) interconnects the rods 120 across the vertebral midline. The transverse connector 100 blocks relative movement of the rods 120 so that the vertebrae V connected to the rods 120 are maintained in their desired relative positions and do not pivot relative to an anterior/posterior axis or a longitudinal central axis of the spinal column, shown as the vertebral midline M. The transverse connector 100 increases the torsional strength of the rod construct to provide stability when the spinal column twists, such as when the shoulders are turned or angled relative to the legs in a standing position. The transverse connector 100 can be located anywhere along the rods 120 and any number of transverse connectors 100 can be used.

FIGS. 2-5 show a first exemplary embodiment of a transverse connector 100. Transverse connector 100 includes a first connecting member 105, a second connecting member 110 and a clamp member 115. The first connecting member 105 includes an integrated clamp portion 125 having a threaded bore 146. The first connecting member 105 also includes an elongate arm 150 extending away from the region of the bore 146. The arm 150 has an elongate opening 144 extending through its middle region and a threaded bore 142 near its terminus opposite bore 146. The second connecting member 110 includes a threaded bore 132 and an elongate arm 160 extending away from the region of bore 132. The arm 160 has an elongate opening 134 extending through its middle region and a threaded bore 136 near its terminus opposite bore 132. The bores do not necessarily have to be threaded. Threads can optionally be used to provide fixation between components of the transverse connector 100.

The second connecting member 110 is removably connectable with clamp member 115 in contrast to the first connecting member 105, which has an integrated clamp portion 125. Clamp member 115 is independent of the second connecting member 110. The second connecting member 110 is connectable with clamp member 115 by way of a threadable clamp screw 112. Threaded bore 132 of the second connecting member 110 can align with a threaded bore 152 of the clamp member 115. Clamp screw 112 threads through aligned bores 132 and 152 thereby fixing the clamp member 115 to the second connecting member 110. Prior to fixing, the clamp 115 can rotate about the axis of the clamp screw 112 relative to the connecting member 110.

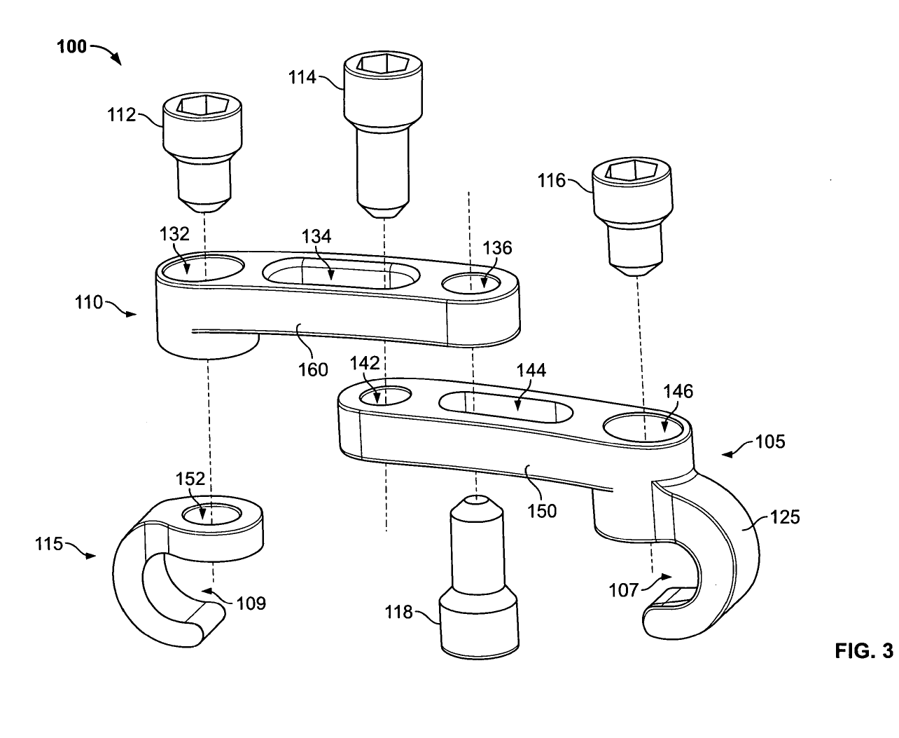

Clamp portion 125 and clamp member 115 each has a recess 107 and 109 (shown in FIG. 3). Recess 107 is sized to removably receive a portion of spine rod 120a. Spine rod 120a is fixed within recess 107 by clamp screw 116, which tightens onto the spine rod 120a. Clamp screw 116 engages the first connecting member 105 through bore 146. Recess 109 is sized to receive a portion of spine rod 120b. Spine rod 120b is fixed within recess 109 by clamp screw 112, which tightens onto the spine rod 120b. Clamp screw 112 engages the second connecting member 110 through threaded bore 132 and clamp member 115 through threaded bore 152, which is aligned with threaded bore 132. Spine rod 120b is fixed within recess 109 by threading clamp screw 112 through aligned bores 132 and 152 of the second connecting member 110 and the clamp member 115, respectively. The spine rods can be fixed within the recesses of clamp members by the clamp screws pressing downward on the top of the rods. The spine rods can also be fixed within the recesses of the clamp members by the clamp screws threading against the side of the spine rods thereby trapping or pressing them within the recesses from the side or top of the spine rods.

As mentioned above, the first connecting member 105 has an elongate arm 150 that extends toward the second connecting member 110 and away from the region of bore 146. The second connecting member 110 similarly has an elongate arm 160 that extends toward the first connecting member 105 and away from the region of bore 132. The elongate opening 144 of arm 150 and elongate opening 134 of arm 160 extend generally transverse to the rods 120a, 120b when the rods 120a, 120b are fixed within the recesses 109, 107 by the clamp screws 116, 112, as described above.

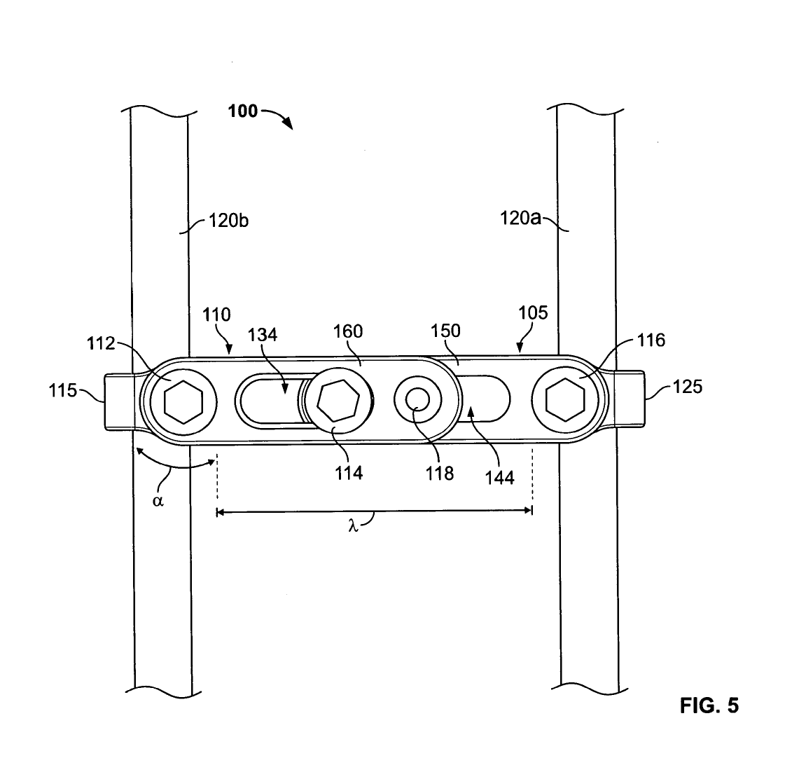

The first connecting member 105 and the second connecting member 110 interconnect, as follows. An upper surface of arm 150 contacts a lower surface of arm 160 such that the second connecting member 110 rests atop the first connecting member 105 along transverse axis, shown in FIG. 5. Thus, the first connecting member 105 lies below the horizontal plane of the second connecting member 110. Threaded bore 142 of arm 150 aligns with at least a portion of elongate opening 134 and threaded bore 136 aligns with at least a portion of elongate opening 144. The overlap of the first and second connecting members 105, 110 is fixed by a pair of set screws 114, 118. Set screw 114 can be threaded from a lower surface of arm 150 through elongate opening 134 into threaded bore 142 of arm 150. Set screw 118 can be threaded from a lower surface of arm 150 through elongate opening 144 into threaded bore 136 of arm 160.

Connecting members 105, 110 are slideably adjustable along transverse axis λ with respect to one another such as in a telescoping manner. The telescoping connecting members 105, 110 can be fixed at a plurality of lengths and into a plurality of positions by set screws 114, 118 as described above. As described above, clamp portion 125 is integral with the first connecting member 105. Second connecting member 110 does not have an integrated clamp mechanism. Instead, clamp member 115 is independent of the second connecting member 110. As best shown in FIG. 5, this configuration allows for rotational adjustment of the second connecting member 110 with respect to the longitudinal member or rod 120b around the axis of clamp screw 112 as shown by arrow α. Transverse and rotational adjustment of connecting members 105, 110 are particularly useful in regions of the spine where the spinal fixation elements implanted therein are not parallel to one another.

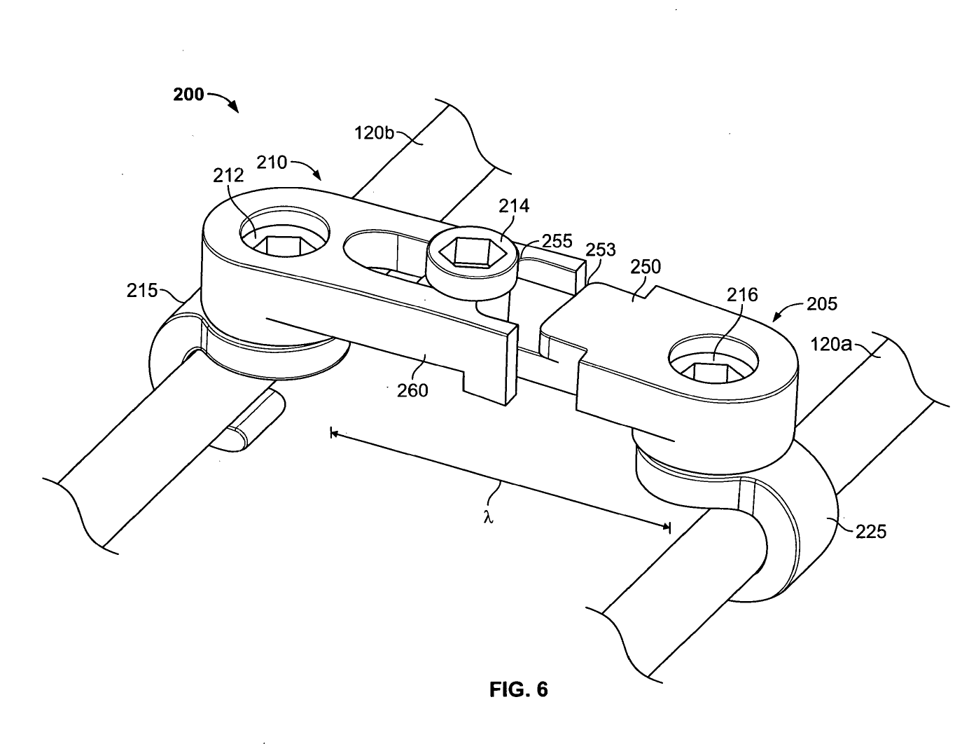

FIGS. 6-9 show another embodiment of a transverse connector 200, in which the first and second connecting members 205, 210 slideably connect by way of a dovetail-like interface. Transverse connector 200 includes a first connecting member 205, a second connecting member 210 and two clamp members 215, 225. The first connecting member 205 includes a threaded bore 246 and an elongate arm 250 that extends away from bore 246. The arm 250 has a threaded bore 242 near its terminus opposite the region of the bore 246. The second connecting member 210 includes a threaded bore 232 and an elongate arm 260 extending away from the bore 232. The arm 260 has an elongate groove 234 that extends through its middle region. The groove 234 has a receiving window 236 at its terminus or the region nearest the vertebral midline when connected to the spinal column. The receiving window 236 is sized and shaped to receive the arm 250 for insertion into the groove 234.

The connecting members 205, 210 are removably connectable with clamp members 225, 215. The first connecting member 205 is connectable with clamp member 225 by way of a threadable clamp screw 216. Threaded bore 246 of the first connecting member 205 can be aligned with a threaded bore 248 of the clamp member 225. Clamp screw 216 threads through the aligned bores 246, 248 thereby fixing the clamp member 225 to the first connecting member 205. The second connecting member 210 is connectable with clamp member 215 by way of a threadable clamp screw 212. Threaded bore 232 of the second connecting member 210 can be aligned with a threaded bore 252 of the clamp member 215. Clamp screw 212 threads through the aligned bores 232, 252 thereby fixing the clamp member 215 to the second connecting member 210.

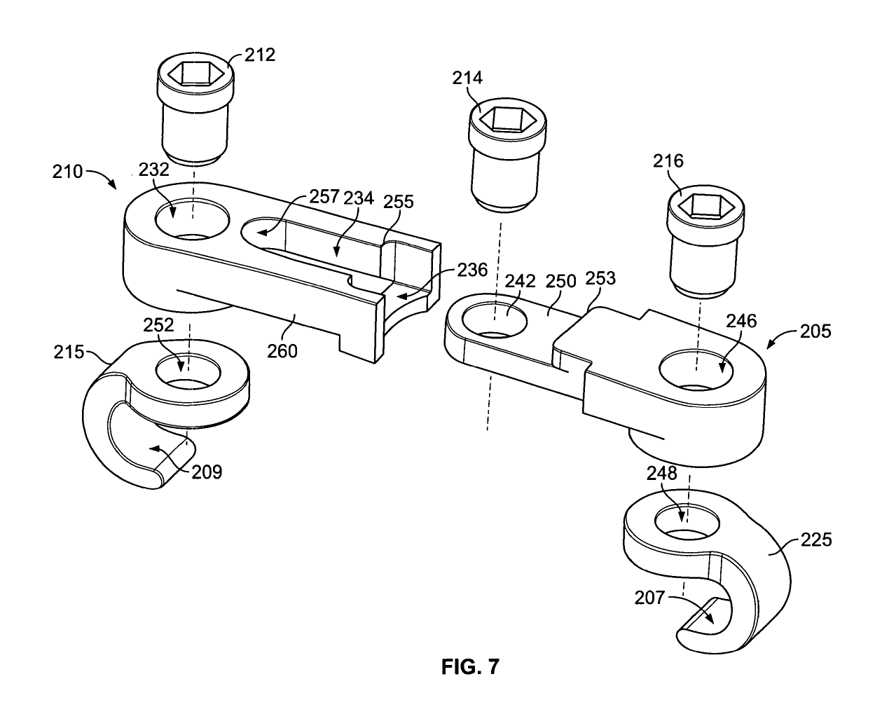

Clamp members 215, 225 each has a recess 209 and 207 (shown in FIG. 7). Recess 207 receives a portion of spine rod 120a. Spine rod 120a is fixed within the recess 207 by clamp screw 216. Clamp screw 216 engages the first connecting member 205 through threaded bore 246 and the clamp member 225 through threaded bore 248, which is aligned with threaded bore 246. Spine rod 120a is fixed within recess 207 by threading clamp screw 216 through aligned bores 246 and 248 of the first connecting member 205 and the clamp member 225, respectively. Recess 209 receives a portion of spine rod 120b. Spine rod 120b is fixed within the recess 209 by clamp screw 212. Clamp screw 212 engages the second connecting member 210 through threaded bore 232 and the clamp member 215 through threaded bore 252, which is aligned with threaded bore 232. Spine rod 120b is fixed within recess 209 by threading clamp screw 212 through aligned bores 232 and 252 of the second connecting member 210 and the clamp member 215, respectively. The spine rods can be fixed within the recesses of clamp members by the clamp screws pressing downward on the top of the rods. The spine rods can also be fixed within the recesses of the clamp members by the clamp screws threading against the top or side of the spine rods thereby trapping or pressing them within the recesses from the side.

As mentioned above, the first connecting member 205 includes an elongate arm 250. Arm 250 extends toward the second connecting member 210 and away from the region of bore 246. The second connecting member 210 similarly includes an elongate arm 260 that extends toward the first connecting member 205 and away from the region of bore 232. Arm 260 has a groove 234 running through it. The arms 250 and 260 extend toward each other in a generally transverse direction to the rods 120a, 120b when the rods 120a, 120b are fixed within the recesses 207, 209 by the clamp screws 216, 212, as described above.

The first connecting member 205 and the second connecting member 210 interconnect by slideably inserting elongate arm 250 inside of elongate arm 260 and engagement by a set screw 214, described as follows. The elongate groove 234 of the second connecting member 210 has openings at the upper and lower surface of the arm 260 as well as a receiving window 236 at the end nearest the vertebral midline. The receiving window 236 is configured to receive arm 250. In this regard, the receiving window 236 can optionally have a shape that corresponds to the shape of the arm 250. For example, if the cross-sectional shape of the arm 250 is rectangular, the shape of the receiving window 236 is likewise rectangular. In an embodiment, the shape of the elongate groove 234 generally mirrors the cross-sectional shape of the arm 250 such that the arm 250 can be inserted through the elongate groove 234 with little resistance. A mirrored or complimentary shape between the groove 234 and the arm 250 provides a smooth interfit that promotes a clean sliding movement between the groove 234 and the arm 250. However, it is not necessary that the shapes be mirrored or complimentary.

The connection between the arm 250 of the first connecting member 205 inside the arm 260 of the second connecting member 210 is fixed by way of a set screw 214. Upon insertion of the arm 250 into the receiving window 236, threaded bore 242 is aligned with at least a portion of the elongate groove 234. The set screw 214 can be threaded from above through an upper opening of the elongate groove 234 into the threaded bore 242. This fixates the first connecting member 205 inside the second connecting member 210 such that the first and second connecting members interlock transversely along the same horizontal plane.

The arm 250 includes a step 253 that abuts a shoulder 255 on the arm 260 as the arm 250 is inserted into the arm 260. The interface between the step 253 and the shoulder 255 provides a stop that limits movement of the arm 250 into the arm 260. The relative positions of the step 253 and shoulder 255 can be selected to provide a predetermined amount of relative sliding movement between the arms 250 and 260. The interface between the screw 214 and an inner wall 257 of the slot 234 can also limit movement between the arms 250 and 260.

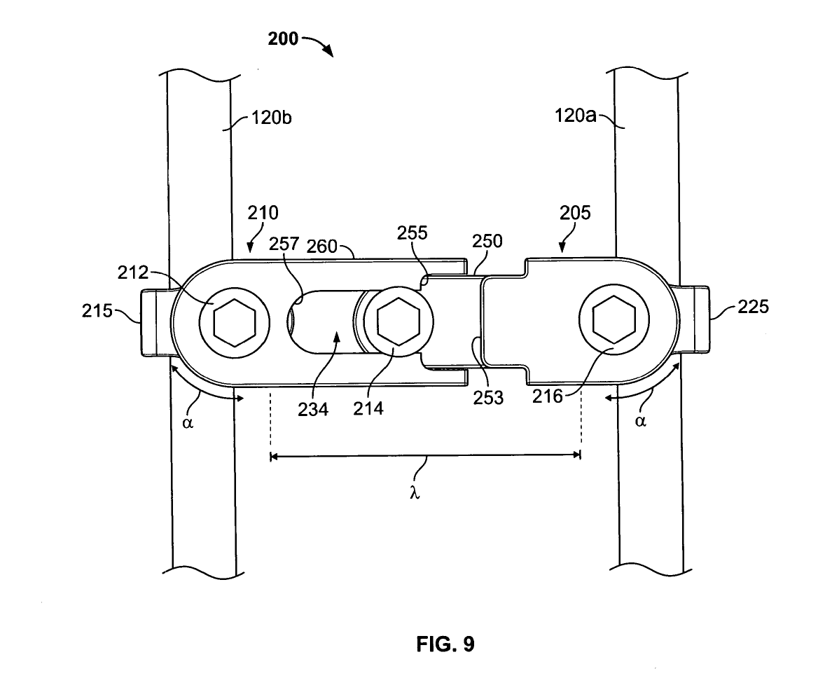

Thus, connecting members 205, 210 are slideably adjustable along transverse axis λ with respect to one another such as in a telescoping manner as shown in FIG. 9. The telescoping connecting members 205, 210 can be fixed at a plurality of lengths and into a plurality of positions by the set screw 214 as described above. In addition, the clamp portions 215 and 225 can be rotationally adjusted relative to the axes of the screws 212 and 216 as shown by arrow α.

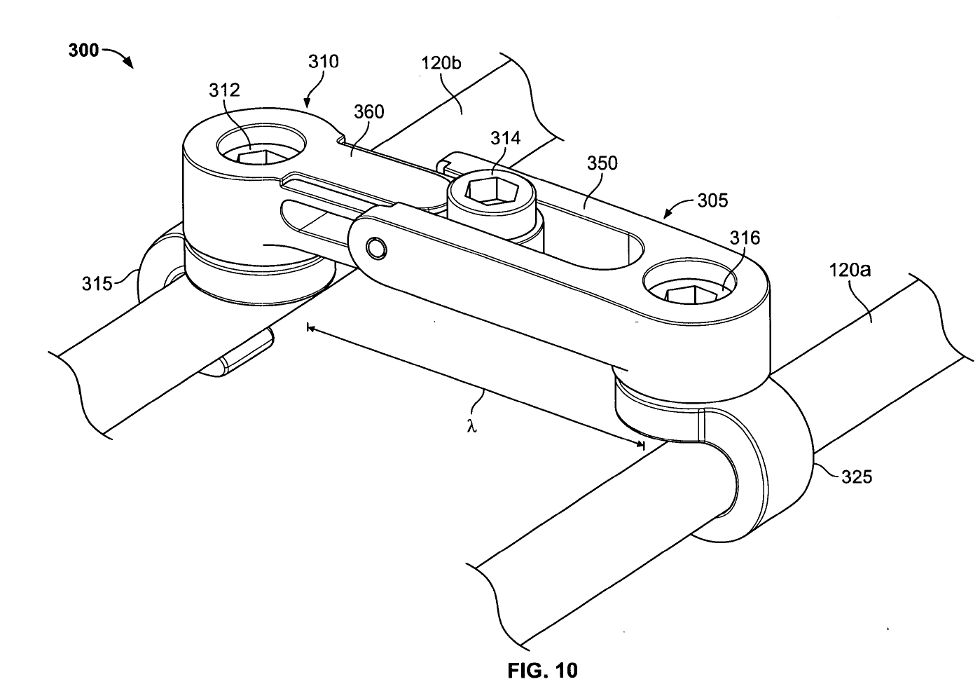

FIGS. 10-13 show another embodiment of a transverse connector 300 in which the first and second connecting members 305, 310 feature a cross pin connection. Transverse connector 300 includes a first connecting member 305, a second connecting member 310 and two clamp members 315, 325. The first connecting member 305 includes a threaded bore 346 and a forked region 350 that extends toward the second connecting member 310 and away from the bore 346. The forked region 350 includes a pair of parallel arms 356a, 356b, at which the terminus of each is a hole 358a, 358b. The second connecting member 310 includes a threaded bore 332 and an elongate arm 360 that extends away from the bore 332 and toward the first connecting member 305. The arm 360 has an elongate slot 334 that extends through its middle region and a threaded bore 336 at the end region located opposite to the region of the threaded bore 332.

The connecting members 305, 310 are removably connectable with clamp members 325, 315. The first connecting member 305 is connectable with clamp member 325 by way of a threadable clamp screw 316. Threaded bore 346 of the first connecting member 305 can be aligned with a threaded bore 348 of the clamp member 325. Clamp screw 316 threads through the aligned bores 346, 348 thereby fixing the clamp member 325 to the first connecting member 305. The second connecting member 310 is connectable with clamp member 315 by way of a threadable clamp screw 312. Threaded bore 332 of the second connecting member 310 can be aligned with a threaded bore 352 of the clamp member 315. Clamp screw 312 threads through the aligned bores 332, 352 thereby fixing the clamp member 315 to the second connecting member 310.

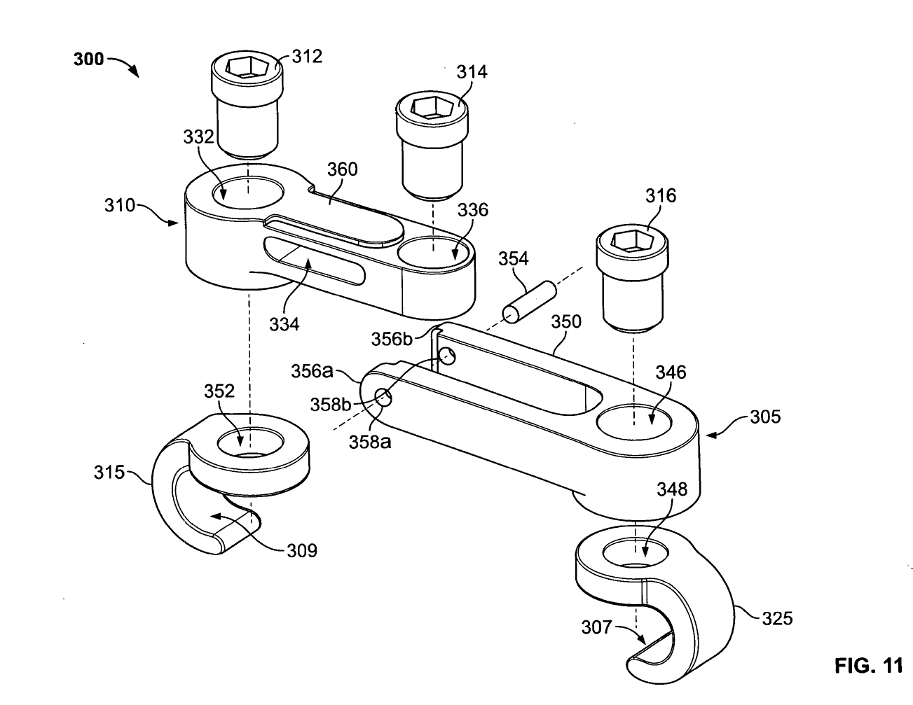

Clamp members 315, 325 each has a recess 309 and 307 (shown in FIG. 11). Recess 307 receives a portion of spine rod 120a. Spine rod 120a is fixed within the recess 307 by clamp screw 316. Clamp screw 316 engages the first connecting member 305 through threaded bore 346 and the clamp member 325 through threaded bore 348, which is aligned with threaded bore 346. Spine rod 120a is fixed within recess 307 by threading clamp screw 316 through aligned bores 346 and 348 of the first connecting member 305 and the clamp member 325, respectively. Recess 309 receives a portion of spine rod 120b. Spine rod 120b is fixed within the recess 309 by clamp screw 312. Clamp screw 312 engages the second connecting member 310 through threaded bore 332 and the clamp member 315 through threaded bore 352, which is aligned with threaded bore 332. Spine rod 120b is fixed within recess 309 by threading clamp screw 312 through aligned bores 332 and 352 of the second connecting member 310 and the clamp member 315, respectively. The spine rods can be fixed within the recesses of clamp members by the clamp screws pressing downward on the top of the rods. The spine rods can also be fixed within the recesses of the clamp members by the clamp screws threading against the side or top of the spine rods thereby trapping or pressing them within the recesses from the side.

As mentioned above, the first connecting member 305 includes a forked region 350 that has parallel arms 356a, 356b extending away from the region of bore 346 toward the second connecting member 310. The second connecting member 310 includes an elongate arm 360 that extends away from the region of bore 332 toward the first connecting member 305. The arms 356a, 356b and arm 360 extend toward each other in a generally transverse direction to the rods 120a, 120b when the rods 120a, 120b are fixed within the recesses 307, 309 by the clamp screws 316, 312, as described above.

The first connecting member 305 and the second connecting member 310 interconnect by positioning elongate arm 360 between parallel arms 356a, 356b of the forked region 350. Arm 360 is positioned between the parallel arms 356a, 356b such that the holes 358a, 358b align with the elongated slot 334. In an embodiment, the shape of arm 360 generally mirrors or complements the shape of the recess of the forked region 350 formed by arms 356a, 356b.

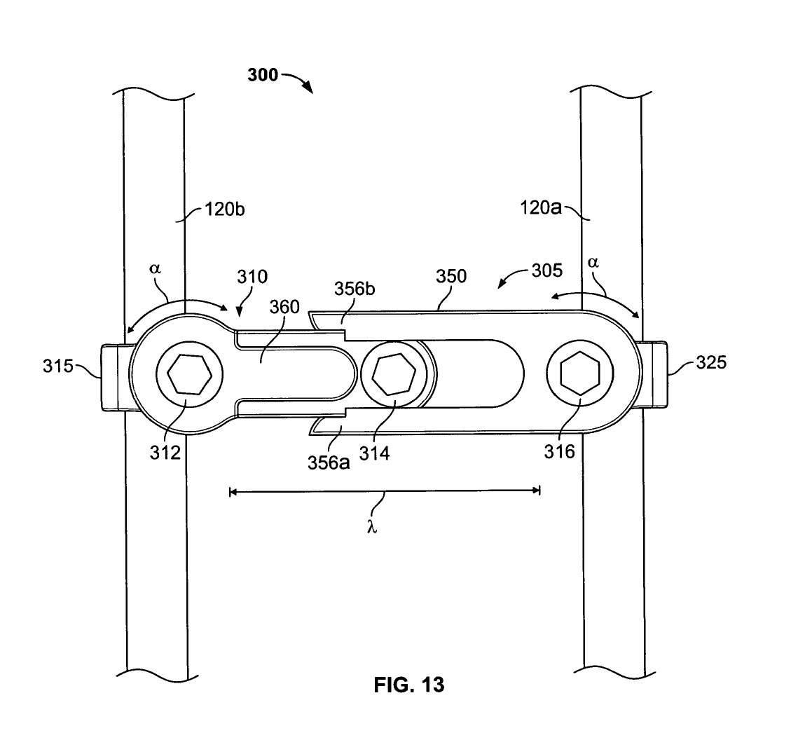

The connection between the first and second connecting members 305, 310 is fixed by way of a set screw 314 and a cross pin 354. Threaded bore 336 extends through the region of the arm 360 located opposite of bore 332. Upon positioning of arm 360 between arms 356a, 356b, holes 358a, 358b are aligned with at least a portion of the elongated slot 334. The cross pin 354 can be inserted through holes 358a, 358b thereby preventing the first connecting member 305 from sliding out of the second connecting member 310. In this manner, the cross pin 354 acts as a detent that limits the amount of telescoping movement (represented by λ in FIG. 13 between the first and second connecting members. Set screw 314 threadably engages the second connecting member 310 through threaded bore 336. The set screw 314 can be tightened to fixate the first and second members relative to one another. In addition, the clamp portions 315 and 325 can be rotationally adjusted relative to the axes of the screws 312 and 316 as shown by arrow α in FIG. 13.

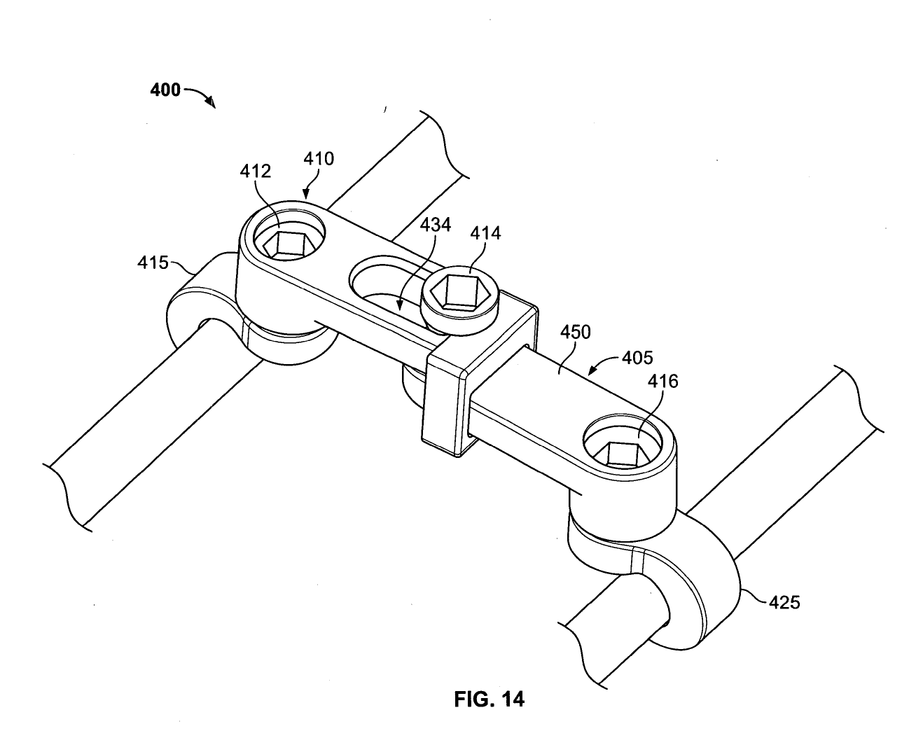

FIGS. 14-17 show another embodiment of a transverse connector 400 in which the first and second connecting members 405, 410 interlock in a free sliding belt-buckle configuration. Transverse connector 400 includes a first connecting member 405, a second connecting member 410 and two clamp members 415, 425. The first connecting member 405 includes a threaded bore 446 and an elongate arm 450. The arm 450 extends away from the bore 446 and toward the second connecting member 410. The arm 450 has a threaded bore 442 at its terminus. The second connecting member 410 includes a threaded bore 432 and an elongate arm 460. The arm 460 extends away from the bore 432 and toward the first connecting member 405. The arm 460 has an elongate slot 434 extending through its middle region and a receiving window 436 located at the end opposite of bore 432. Further, the receiving window 436 lies below the horizontal plane of the arm 460.

The connecting member 405, 410 are connectable with clamp members 325, 315. The first connecting member 405 is connectable with clamp member 425 by way of a threadable clamp screw 416. Threaded bore 446 of the first connecting member 405 can be aligned with a threaded bore 448 of the clamp member 425. Clamp screw 416 threads through the aligned bores 446, 448 thereby fixing the clamp member 425 to the first connecting member 405. The second connecting member 410 is connectable with clamp member 415 by way of a threadable clamp screw 412. Threaded bore 432 of the second connecting member 410 can be aligned with a threaded bore 452 of the clamp member 415. Clamp screw 412 threads through the aligned bore 432, 452 thereby fixing the clamp member 415 to the second connecting member 410.

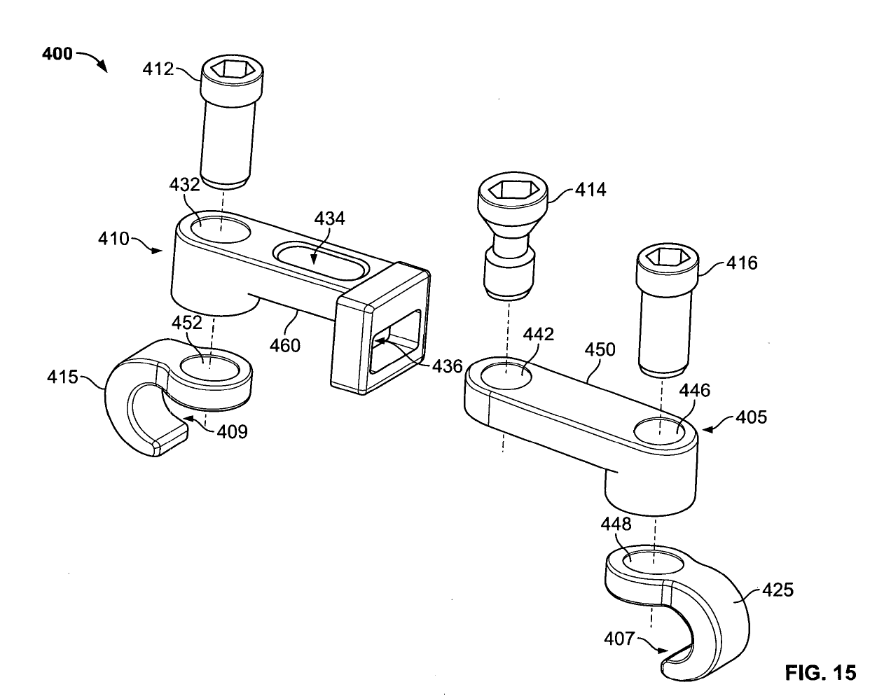

Clamp members 415, 425 each has a recess 409 and 407 (shown in FIG. 15). Recess 407 receives a portion of spine rod 120a. Spine rod 120a is fixed within the recess 407 by clamp screw 416. Clamp screw 416 engages the first connecting member 405 through threaded bore 446 and the clamp member 425 through threaded bore 448, which is aligned with threaded bore 446. Spine rod 120a is fixed within recess 407 by threading clamp screw 416 through aligned bores 446 and 448 of the first connecting member 405 and the clamp member 425, respectively. Recess 409 receives a portion of spine rod 120b. Spine rod 120b is fixed within the recess 409 by clamp screw 412. Clamp screw 412 engages the second connecting member 410 through threaded bore 432 and the clamp member 415 through threaded bore 452, which is aligned with threaded bore 432. Spine rod 120b is fixed within recess 409 by threading clamp screw 412 through aligned bores 432 and 452 of the second connecting member 410 and the clamp member 415, respectively. The spine rods can be fixed within the recesses of clamp members by the clamp screws pressing downward on the top of the rods. The spine rods can also be fixed within the recesses of the clamp members by the clamp screws threading against the side or top of the spine rods thereby trapping or pressing them within the recesses from the side.

As mentioned above, the first connecting member 405 includes an elongate arm 450 that extends away from the region of bore 446 toward the second connecting member 410. The second connecting member includes an elongate slot 434 extending through its middle region and a receiving window 436 located below the horizontal plane of the arm 460. Arms 450 and 460 extend toward each other in a generally transverse direction to the rods 120a, 120b when the rods 120a, 120b are fixed within the recesses 407, 409 by the clamp screws 416, 412, as described above.

The first connecting member 405 and the second connecting member 410 interconnect by slidably inserting elongate arm 450 through the receiving window 436 of arm 460 and engagement by a set screw 414, described as follows. The receiving window 436 is configured to receive arm 450 of the first connecting member 405. In this regard, the window 436 has a size that is larger than the cross-sectional size of the arm 450 such that the arm 450 is insertable into the window 436. The window 436 can optionally have a shape that complements or mirrors the cross-sectional shape of the arm 450. For example, if the cross-sectional shape of the arm 450 is rectangular, the shape of the window 436 is likewise rectangular. The shape of the window 436 can generally mirror the cross-sectional shape of arm 450 such that the arm 450 can be inserted through the window 436 with little resistance.

The connection between the arm 450 of the first connecting member 405 inside the arm 460 of the second connecting member 410 is fixed by way of a set screw 414. Upon insertion of the arm 450 through the receiving window 436 of arm 460, threaded bore 442 is aligned with at least a portion of the elongate slot 434. Because the window 436 is positioned below the horizontal plane of arm 460, the upper surface of the arm 450 contacts the bottom surface of arm 460. The set screw 414 can be threaded from above through an upper opening of the elongate slot 434 into the threaded bore 442. This fixates the first connecting member 405 through the receiving window 436 and under the second connecting member 410 such that the first and second connecting members interlock transversely.

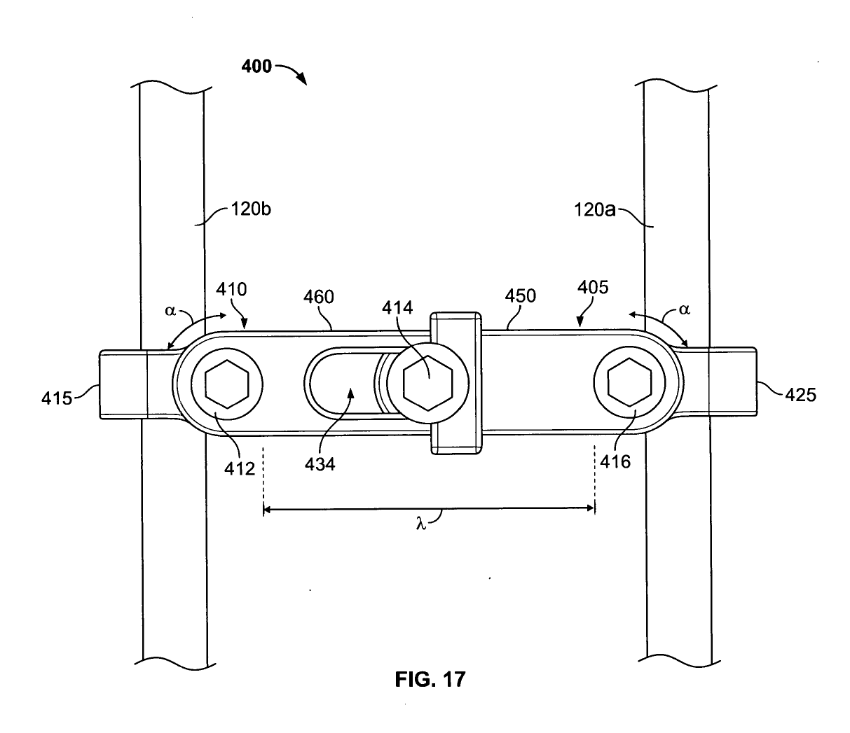

When connected, the first and second members 410 and 405 can move relative to one another in a telescoping manner as limited by the set screw 414 abutting the edges of the slot 434. In addition, the clamp portions 415 and 425 can be rotationally adjusted relative to the axes of the screws 412 and 416 as shown by arrow α in FIG. 17.

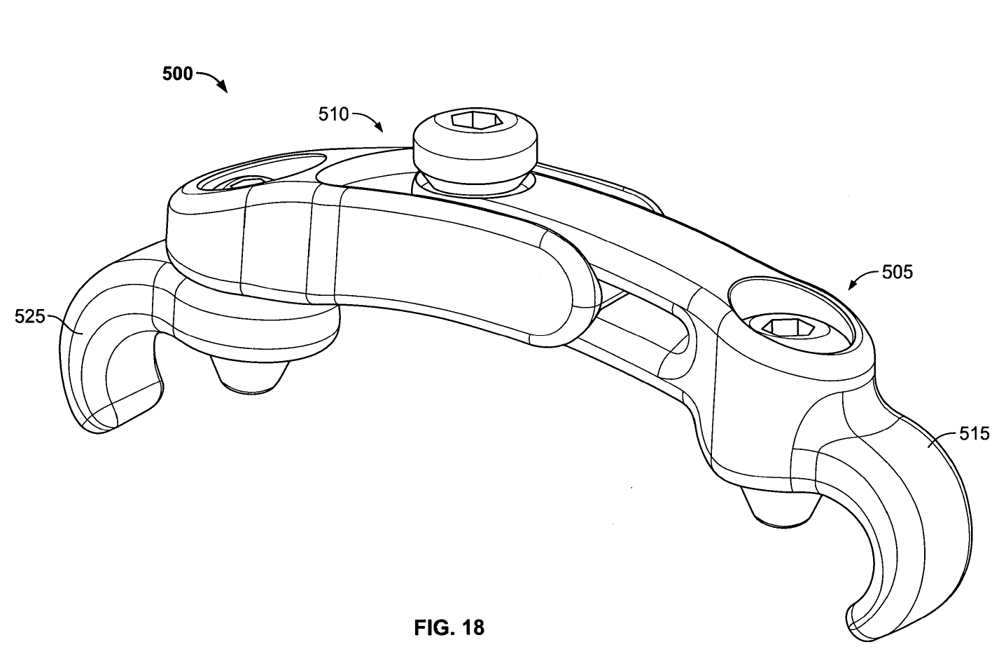

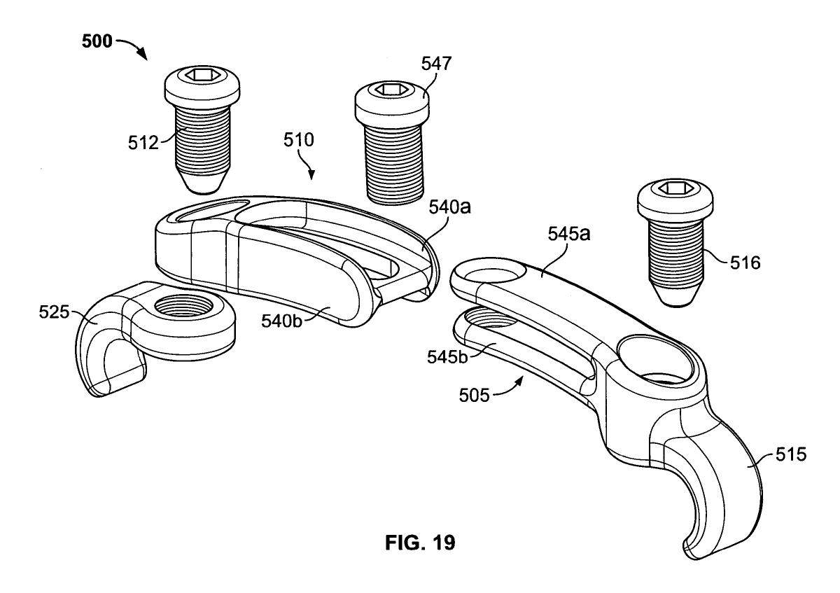

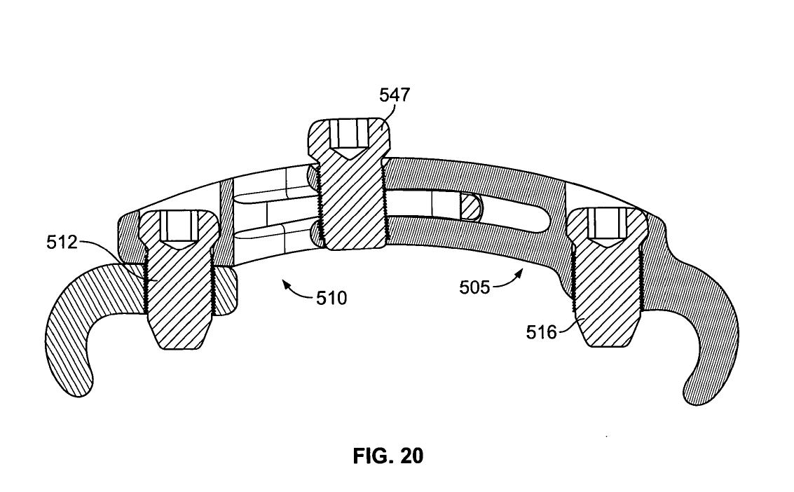

FIGS. 18-20 shows another embodiment of a transverse connector 500 in which the first and second connecting members 505, 510, respectively, are slidably connected to one another. An end of the connecting member 505 has a clamp member 515 that is integrally or monolithically formed with the connecting member 505. The clamp member 515 has a recess that is sized and shaped to receive an elongate rod therein. In this regard, a clamp screw 516 couples to a bore in the connecting member 505 such as in a threaded relationship. The clamp screw 516 can be threaded downward toward a rod positioned within the cavity to tighten against the rod and lock it in the cavity.

An end of the connecting member 510 also has a clamp member 525, which is removably attached to the connecting member 510. The clamp member 525 has a recess that is sized and shaped to receive an elongate rod therein. A clamp screw 512 couples to a bore in the connecting member 510 such as in a threaded relationship. The clamp screw 512 can be threaded downward toward a rod positioned within the cavity to tighten against the rod and lock it in the cavity. The clamp screw 512 is also used to removably secure the clamp member 525 to the connecting member 510.

With reference to FIGS. 18-20, the connecting member 510 is formed of a pair of forked arms 540a and 540b (collectively arms 540) that define a space therebetween. A stop member 541 interconnects the arms 540 at a predetermined location to serve as detent or stop that limits slidable movement between the connecting members 510 and 505, as described more fully below. The connecting member 505 also has a pair of forked arms 545a and 545b (collectively arms 545) that form a space therebetween, wherein the space is aligned along a plane that is normal to a plane defined by the space between the arms 540. A pair of aligned boreholes extend through the arms 545 for receipt of a lock screw 547. The lock screw 547 can be threaded or otherwise engaged into the boreholes to provide a locking force that locks the arms 545 in a fixed position relative to the arms 540.

As shown in FIG. 18, the arms 540 can mate with the arms 545 such that the connecting member 510 is slidably engaged with the connecting member 505. The arms 540 and 545 serve as a sliding interconnection that permits sliding, relative movement between the first and second connecting members. The space between the arms 540 is aligned with the space between the arms 545 such that the arms are adjacent to one another. The locking screw 547 can be tightened downward to provide a compressive force between the arms 545 such that the arms 545 compress the arms 540 and lock the connecting members in a fixed position relative to one another.

Although embodiments of various methods and devices are described herein in detail with reference to certain versions, it should be appreciated that other versions, embodiments, methods of use, and combinations thereof are also possible. Therefore the spirit and scope of the appended claims should not be limited to the description of the embodiments contained herein.

1. A transconnector for connecting first and second spinal rods that are positioned longitudinally along a spine, comprising:
a first member having a first clamp member adapted to clamp onto a first spinal rod, the first member having a first connecting region extending away from the clamp member;
a second member having a second clamp member adapted to clamp onto a second spinal rod, the second member having a second connecting region extending away from the second clamp member toward the first member, wherein the second member and first member are slidably interconnected along the first and second connecting regions;
a first interference pin coupled to the first clamp member and adapted to provide an interfering engagement with the first spinal rod to lock the first spinal rod in the first clamp member;
a second interference pin coupled to the second clamp member and adapted to provide an interfering engagement with the second spinal rod to lock the second spinal rod in the second clamp member; and
a third interference pin coupled to the first and second connecting regions and adapted to provide an interfering engagement between the first and second connecting regions to lock the first and second members in a fixed position relative to one another.

2. A transconnector as in claim 1, wherein the first clamp member is removably attached to the first member.

3. A transconnector as in claim 2, wherein the first interference pin removably attaches the first clamp member to the first member.

4. A transconnector as in claim 1, wherein the first connecting region comprises a pair of forked arms, and wherein the second connecting region comprises a pair of forked arms, and wherein the forked arms of the first connecting region are oriented at about 90 degrees relative to the forked arms of the second connection region.

5. A transconnector as in claim 1, wherein the third interference pin clamps the forked arms of the first connecting region around the forked arms of the second connecting region.

6. A transconnector as in claim 1, wherein at least one of the first, second, and third interfering pins is threaded.

7. A transconnector as in claim 1, wherein the first connecting member has a limited range of sliding motion relative to the second connecting member.

8. A transconnector as in claim 1, wherein the first member can rotate relative to the second member.

9. A transconnector as in claim 1, wherein the first connecting member and the second connecting member can rotate relative to one another about two separate axes of rotation.

10. A transconnector for connecting first and second spinal rods that are positioned longitudinally along a spine, comprising:
a first member having a first clamp member adapted to clamp onto a first spinal rod and a first pair of forked arms extending from the clamp member, wherein the first pair of forked arms extends in a cantilevered manner away from the first clamp member;
a second member having a second clamp member adapted to clamp onto a second spinal rod and a second pair of forked arms extending from the second clamp member, the second pair being configured to slidingly receive the first pair within a space between the second pair, wherein each of the arms in the second pair of forked arms has an end disconnected from the end of the other forked arm at the respective end of each forked arm, wherein the second pair of forked arms extends in a cantilevered manner away from the second clamp member; and
a locking member coupled to an end portion of the first pair and configured to lock the first and second members in a fixed relative position by compressing the first pair together, thereby engaging a portion of the second pair.

11. A transconnector as in claim 10, wherein, when the first pair is received into the space, the first member has a limited range of sliding motion relative to the second member.

12. A transconnector as in claim 10, wherein an end portion of the second pair comprises a stop member that extends between each of the forked arms.

13. A transconnector as in claim 12, wherein, when the first and second members are coupled together, the stop member limits relative sliding motion between the first and second connecting members by engaging the locking member.

14. A transconnector as in claim 10, wherein the locking member comprises a locking screw.

15. A transconnector as in claim 10, wherein the first pair extends in an orientation normal to that of the second pair when the first and second members are coupled together.

16. A transconnector for connecting first and second spinal rods that are positioned longitudinally along a spine, comprising:
a first member having (a) a first clamp member, configured to engage a first spinal rod, and (b) a first pair of forked arms extending from the first clamp member in a first orientation, wherein the first pair of forked arms extends in a cantilevered manner away from the first clamp member;
a second member having (a) a second clamp member, configured to engage a second spinal rod, and (b) a second pair of forked arms extending from the second clamp member in a second orientation normal to the first orientation when the first and second clamp members are coupled together, the second pair being configured to slidingly receive the first pair in a space defined by the second pair, wherein each of the arms in the second pair of forked arms has an end disconnected from the end of the other forked arm at the respective end of each forked arm, wherein the second pair of forked arms extends in a cantilevered manner away from the second clamp member; and
a locking member coupled to an end portion of the first pair and configured to lock the first and second members in a fixed relative position by compressing the first pair together, thereby engaging a portion of the second clamp between the second pair.

17. A transconnector as in claim 16, wherein the locking member comprises a locking screw.

18. A transconnector as in claim 16, wherein, when the first pair is received into the space, the first member has a limited range of sliding motion relative to the second member.

19. A transconnector as in claim 16, wherein an end portion of the second pair comprises a stop member that extends between each of the forked arms.

20. A transconnector as in claim 16, wherein, when the first and second members are coupled together, the stop member extending between an end portion of the second pair limits relative sliding motion between the first and second connecting members.
